(19) United States
(12) Patent Application Publication
(10) Pub. No.: US 2014/0024992 A1
(43) Pub. Date: Jan. 23, 2014

(54) Cylindrical Structure Having Lumen to Be Implanted Into Human Body

(71) Applicants: Eun-Jin Kim, Seongnam-si (KR); In-Su Baek, Anyang-si (KR); Ji-Hye Park, Incheon (KR); Dae-Joong Kim, Seongnam-si (KR)

(72) Inventors: Eun-Jin Kim, Seongnam-si (KR); In-Su Baek, Anyang-si (KR); Ji-Hye Park, Incheon (KR); Dae-Joong Kim, Seongnam-si (KR)

(73) Assignee: M.I. TECH CO., LTD., Gyeonggi-do (KR)

(21) Appl. No.: 14/007,645

(22) PCT Filed: Jul. 10, 2012
(86) PCT No.: PCT/KR2012/005444

§ 371 Date: Sep. 25, 2013

(51) Int. Cl. A61M 27/00

(52) U.S. Cl. CPC A61M 27/002
USPC 604/8; 427/2.25

(57) ABSTRACT

A cylindrical structure having a lumen to be implanted into a human body. The cylindrical structure has a coating layer formed on at least one part of an inner surface of either or both ends of the cylindrical structure, wherein the coating layer includes a polymer for drug release control and a bioactive material.

### TECHNICAL FIELD

The present invention relates to a cylindrical structure having a lumen to be implanted into the human body, and, more particularly, to a cylindrical structure having a lumen, which is configured to be implanted into the human body, functioning as a connector between blood vessels, or as a stable approach channel between the artery and the vein in the human body, particularly, of hemodialysis patients, by which the occurrence of angiogenesis is greatly reduced,

### BACKGROUND ART

When several problems, such as stenosis, occur in luminal organs (blood vessels, esophagi, intestines, and the like) of the human body, a cylindrical structure having a lumen is often used to achieve the desired medical outcome. In this case, however, the connection part between the implanted structure and the human body may cause various problems, such as stenosis, occlusion, and inflammation, etc.

For example, when blood supply is restricted due to angiostenosis, a coronary artery disease or a peripheral vessel disease may result. For the treatment of the stenosis-induced peripheral vessel disease, a bypass graft is provided, or an artificial blood vessel is implanted after excising the occluded part, so as to remove the stenosis. Even in these cases, however, problems may occur with the graft, such as stenosis between the graft and the blood vessel, and inflammation around the graft.

Meanwhile, patients with severe renal insufficiency are generally treated by hemodialysis and the number of patients undergoing this procedure has been increasing, Generally, most patients in need of hemodialysis suffer from diabetes and hypertension, in addition to having severe arteriosclerosis. However, in order to conduct effective hemodialysis, there must be no disturbances against the flow of blood between the artery and the vein for a long period of time. For this purpose, extensive research has been conducted.

As a typical example, there is an artificial blood vessel. An artificial blood vessel has been developed as an alternative means to guide the flow of blood when a patient's blood vessel is narrowed by any cause or its function is remarkably deteriorated.

FIG. 1 shows an artificial blood vessel 10 connecting the artery 12 and vein 14 of a patient under hemodialysis. An arteriovenous fistula operation starts with incision of the patient to the hypordermis at a predetermined body region, followed by perforating the artery and the vein, both of which are exposed, and then by joining opposite ends of an artificial vessel to the perforated vessels.) Like a capillary between the artery and the vein, the artificial vessels placed and sutured in the incised region function as a path through which blood flows during hemodialysis, with the communication of a hemodialyzer with the artery and the vein via a needle connected thereto.

An artificial blood vessel is both advantageous and disadvantageous compared to an arteriovenous fistula used to allow a hemodialyzed patient to use his own blood vessel to undergo hemodialysis. in particular, the artificial blood vessel is problematic in that angiostenosis or thrombosis occurs at the connection between the artificial blood vessel and the artery and vein, Thus, a solution for this problem is required.

A study indicated that stenosis at the connection point between the artificial blood vessel and the artery and/or the vein is caused by the neointimal hyperplasia of vascular tissues. Many attempts have been made to develop an artificial blood vessel that is configured to regulate the neointimal hyperplasia of vascular tissues so as to prevent antigostenosis.

### DISCLOSURE

### Technical Problem

The present invention intends to solve the problems occurring when a cylindrical structure having a lumen is implanted in the human body, and, particularly, to solve the problems of stenosis, occlusion and inflammation at the connection between the implanted cylindrical structure and the human body. An object of the present invention is to provide a cylindrical structure having a lumen to he implanted into the human body, which can stably connect the artery and vein of a patient undergoing periodic hemodialysis and which can greatly reduce the occurrence of angiostenosis.

### Technical Solution

In order to accomplish the above object, an aspect of the present invention provides a cylindrical structure having a lumen to be implanted into a human body, including: a coating layer partially formed on an inner surface of either or both ends thereof, wherein the coating layer includes a polymer for drug release control and a bioactive material.

### Advantageous Effects

According to the present invention, the problems occurring when a cylindrical structure having a lumen is implanted in the human body, inter alia, stenosis, occlusion and inflammation at the connection between the implanted cylindrical structure and the body can be solved). In particular, when a hemodialyzed patient cannot use his own blood vessel as a blood channel for hemodialysis at the time of hemodialysis, the cylindrical structure of the present invention can be used to connect the artery and the vein to allow for the stable performance of hemodialysis, and can actively control the neointimal hyperplasia of blood vessel tissues at the connection) between the cylindrical structure and the artery and vein in the human body because this cylindrical structure contains an anticancer agent or an inflammation inhibitor.

Further, the cylindrical structure of the present invention can prevent edema or stenosis at the connection between the implanted cylindrical structure and the artery and vein in the human body can be prevented, thus making a great contribution to a reduction in cost as well as reducing patients' pain.

### DESCRIPTION OF DRAWINGS

FIG. 1 is a schematic view showing a connection between the artery and the vein in a patient under hemodialysis.

FIG. 2 is a sectional view showing a cylindrical structure having a lumen to be implanted into the human body according to an embodiment of the present invention.

FIG. 3 is a sectional view showing a cylindrical structure having a lumen to be implanted into the human body according to another embodiment of the present invention.

FIG. 4 is a schematic view showing a method of manufacturing a cylindrical structure having a lumen to be implanted into the human body according to an embodiment of the present invention.

FIG. 5 is a schematic view showing a method of manufacturing a cylindrical structure having a lumen to be implanted into the human body according to another embodiment of the present invention.

FIG. 6 is a graph comparing the drug release patterns of artificial blood vessels of Example and Comparative Examples 1 to 3.

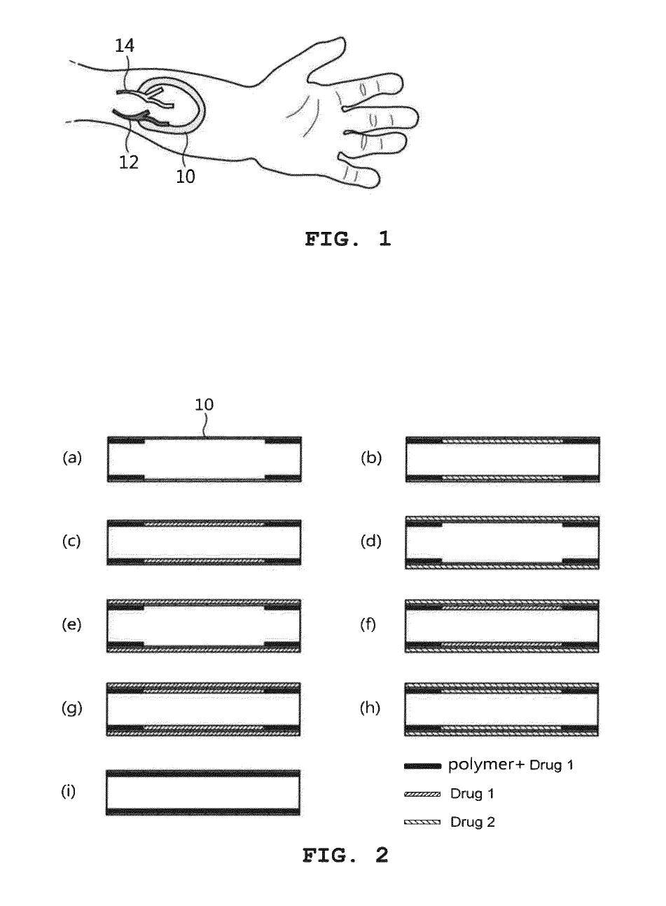

### BEST MODE

Hereinafter, preferred embodiment of the present invention will be described in detail with reference to the accompanying drawings.

The present invention provides a cylindrical structure having a lumen to be implanted into the human body, which can prevent the connection between the cylindrical structure and the artery and vein in the human body from being stenozed by neointimal hyperplasia.

Specifically, the present invention provides a cylindrical structure having a lumen to be implanted into a human body, including: a coating layer formed on at least one part of an inner surface of either or both ends of the cylindrical structure, wherein the coating layer includes a polymer for drug release control and a bioactive material.

The cylindrical structure may be made of e-PTFE (expanded poly-tetrafluoroethylene), polyethylene erephthalate, polyurethane, polyamide, polyester, polyolefin or the like.

Among these polymers, e-PTFE (expanded polytetrafluoroethylene) is advantageous because it is formed into a thin film having micropores by extruding PTFE at high temperature and high pressure and then expanding the extruded PTFE in various ways and because this thin film retards the adsorption of protein thereon thanks to its low friction coefficient to improve antithrombogenicity.

The cylindrical structure of the present invention is characterized in that a coating layer including a polymer for drug release control and a bioactive material is at least partially formed on an inner surface of either or both ends thereof.

The coating layer is formed by applying a mixed solution including a polymer for drug release control and a bioactive material onto inner surfaces of both ends of the cylindrical structure having a lumen to be implanted into the human body.

It is preferred that the length of the coating layer from both ends of the cylindrical structure be 2˜15 cm.

Examples of the bioactive materials may include: antiproliferative/anticancer agents such as paclitaxel, methotrexate and the like; immune suppressors such as rapamycin, tacrolimus, cyclosporine A and the like; and steroid or non-steroid anti-inflammatory agents such as dexamethasone, dexamethasone phosphate, dexamethasone acetate and the like. These bioactive materials may be used independently or in a mixture thereof. The bioactive material is not limited thereto as long as it is a drug that can suppress the occurrence of stenosis or inflammation of blood vessels. Preferably, paclitaxel or rapamycin may be used as the bioactive material.

It is preferred that the capacity of the bioactive material included in the cylindrical structure per unit area is 0.1˜3 μg/mm².

The polymer for drug release control included in the mixed solution serves to allow a drug for suppressing neointimal hyperplasia to be absorbed in cells adjacent to a blood vessel all in an optimally effective amount over the entire critical period having a danger of blood vessel restenosis by continuously releasing the drug for suppressing neointimal hyperplasia.

The polymer for drug release control is a biologically-stable and biodegradable polymer or polysaccharide that can be used as a carrier or matrix of a drug. Examples of the polymer for drug release control may include, but are not limited to, collagen, cellulose acetate, hyaluronic acid, polylactic acid, polyglyeolic acid, polyanhydride, polycaprolactone or copolymers or compounds thereof.

The concentration of the polymer for drug release control included in the mixed solution may be 0.01˜10 mg/mL (w/v).

The mixed solution may further include a mixed solvent in which solvents having different penetrability to the cylindrical structure from each other are mixed.

For example, the mixed solvent may include a polar solvent and a nonpolar solvent at a predetermined ratio to have suitable polarity.

In this case, a nonpolar solvent can be used if it has high volatility and can easily dissolve a drug. As a nonpolar solvent, acetone, diethyl ether, ethyl acetate, dichloromethane, n-haxane, chloroform, teat-butanol or the like may be used according to the solubility of a drug. In particular, since acetone has high volatility, when a drug-dissolved coating solution permeates into a cylindrical structure having a lumen to be implanted into the human body, acetone is easily volatilized from the coating solution, and thus only the drug is applied on the inner surface of the cylindrical structure.

Further, in this case, it is preferred that water be used as a polar solvent, acetone be used as a nonpolar solvent, and a ratio of acetone to water be 7:3˜9:1.

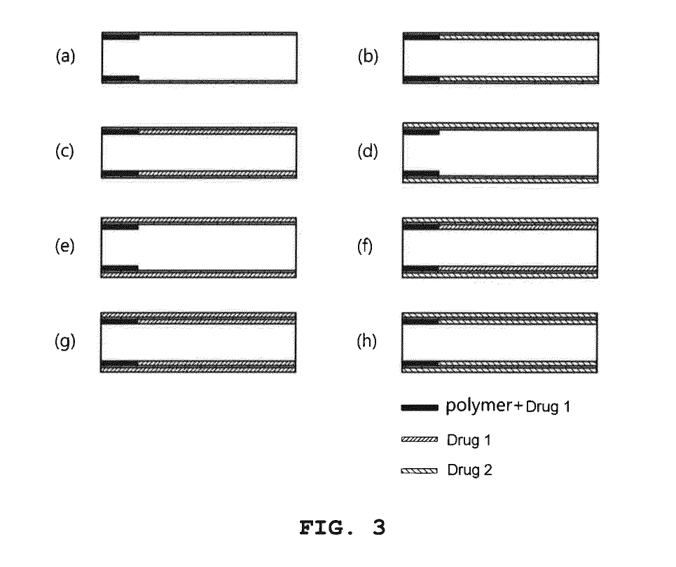

An embodiment of the present invention provides a cylindrical structure having a lumen to be implanted into a human body, including: a coating layer containing a polymer for drug release control and a bioactive material and partially formed on an inner surface of either or both ends thereof (refer to FIG. 2a and FIG. 3a).

The present inventors found that, even when a coating layer containing a polymer for drug release control and a bioactive material is partially formed on an inner surface of either or both ends of a cylindrical structure having a lumen to be implanted into a human body, the occurrence of angiostenosis and thrombosis due to neointimal hyperplasia is reduced at the venous anastomosis site of an artery-vein-transplanted patient under hemodialysis.

Another embodiment of the present invention provides a cylindrical structure having a lumen to be implanted into a human body, including: a first coating layer including a polymer for drug release control and a bioactive material and partially formed on an inner surface of either or both ends thereof; and a second coating layer including a bioactive material and a drug for inflammatory reaction control and formed on the inner surface thereof, this inner surface not being provided with the first coating layer (refer to FIGS. 2b and 2c and FIGS. 3b and 3c).

The purpose of additionally forming the second coating layer on the non-coated inner surface of the cylindrical structure is to reduce the occurrence of angiostenosis and thrombosis over the entire inner surface of the cylindrical structure including the arteriovenous anastomosis site of an artery-vein-transplanted patient under hemodialysis. Angiostenosis and thrombosis occurring around the implanted cylindrical structure generally occurs at the connection between the implanted cylindrical structure and a cylindrical structure having a lumen in the human body, but are not limited thereto. Therefore, the additionally-formed second coating layer will be effective at preventing the angiostenosis and thrombosis occurring at a connection other than connection. Further, the second coating layer may be effective at preventing a reaction occurring at the junction between the implanted cylindrical structure and another human body, nota reaction occurring at the connection between the implanted cylindrical structure and a structure having a lumen in a human body, particularly, an inflammatory reaction.

Examples of the drugs for inflammatory reaction control may include: antiproliferative/anticancer agents such as paclitaxel, methotrexate and the like; immune suppressors such as rapamycin, tacrolimus, cyclosporine A and the like; and steroid or non-steroid anti-inflammatory agents such as dexamethasone, dexamethasone phosphate, dexamethasone acetate and the like. These drugs for inflammatory reaction control may be used independently or in a mixture thereof. These drugs for inflammatory reaction control may be used without limitation as long as they are drugs that can suppress the occurrence of inflammation of blood vessels.

The bioactive material used in the cylindrical structure may be the same as or different from this drug for inflammatory reaction control.

Still another embodiment of the present invention provides a cylindrical structure having a lumen to be implanted into a human body, including: a first coating layer including a polymer for drug release control and a bioactive material and partially formed on an inner surface of either or both ends of the cylindrical structure; and a second coating layer including a bioactive material and a drug for inflammatory reaction control and formed on an outer surface of the cylindrical structure (refer to FIGS. 2d and 2e and FIGS. 3d and 3e). A reaction occurring at the junction between the implanted cylindrical structure and another human body, not a reaction occurring in the connection between the implanted cylindrical structure and a structure having a lumen in a human body, particularly, an inflammatory reaction, generally occurs at the outer side of a lumen compared to at the inner side thereof. The reason for this is that cells or substances causing an inflammatory reaction using blood flow are diluted. Therefore, it is more effective to form a second coating layer for controlling an inflammatory reaction on the outer surface of the cylindrical structure.

According to the present invention, a coating layer including a bioactive material and a drug for inflammatory reaction control is additionally formed on the outer surface of the cylindrical structure having a lumen to be implanted into the human body, thus reducing the occurrence of an inflammation due to the cylindrical body.

Still another embodiment of the present invention provides a cylindrical structure having a lumen to be implanted into a human body, including: a first coating layer including a polymer for drug release control and a bioactive material and partially formed on an inner surface of either or both ends of the cylindrical structure; and a second coating layer including a bioactive material and a drug for inflammatory reaction control and formed on the inner surface of the cylindrical structure, this inner surface being not provided with the first coating layer; and a third coating layer including a bioactive material and a drug for inflammatory reaction control and formed on the outer surface of the cylindrical structure (refer to FIGS. 2f, 2g and 2h and FIGS. 3f, 3g and 3h).

Still another embodiment of the present invention provides a cylindrical structure having a lumen to be implanted into a human body, including: a coating layer including a polymer for drug release control and a bioactive material and formed on the entire inner surface of the cylindrical structure (refer to FIG. 2i).

Still another embodiment of the present invention provides a cylindrical structure having a lumen to be implanted into a human body, including: a coating layer formed on at least one part of an inner surface of either or both ends of the cylindrical structure, wherein the coating layer includes a bioactive material.

Each of the first, second and third coating layers is formed on the cylindrical structure having a lumen to be implanted into the human body using at least one method selected from the group consisting of perfusion, spraying and dipping.

Specifically, in a method of partially forming a coating layer including a polymer for drug release control, a bioactive material and a mixed solvent on an inner surface of either or both ends of the cylindrical structure, one end of the cylindrical structure is placed on a horizontal plane, and the other end thereof is inclined upwards at a predetermined angle, and then a mixed solution including a polymer for drug release control, a bioactive material and a mixed solvent is perfused by a syringe or a peristaltic pump.

Particularly, in the method, the cylindrical structure formed into a J-shaped L-shaped cylindrical structure, the mixed solution is upwardly perfused from one end of the J-shaped or L-shaped cylindrical structure to the other end thereof, and then the mixed solution is discharged after predetermined time to coat a part of both ends of the J-shaped or L-shaped cylindrical structure with the mixed solution. In the present invention, such a coating method is referred to as “J dipping” or “L dipping”.

Alternatively, the cylindrical structure is vertically placed, and is then sprayed with a mixed solution including a polymer for drug release control, a bioactive material and a mixed solvent in a direction from the bottom of the cylindrical body toward the top thereof by a sprayer.

Meanwhile, in a method of forming a coating layer including a bioactive material or a drug for inflammatory reaction control on the entire outer surface of the cylindrical structure, both ends of the cylindrical structure are sealed, and then the sealed cylindrical structure is dipped into a dipping tank containing a bioactive material or a drug for inflammatory reaction control. Alternatively, the coating layer may be formed on the entire outer surface of the cylindrical structure by spraying a bioactive material or a drug for inflammatory reaction control onto the entire outer surface thereof using a sprayer.

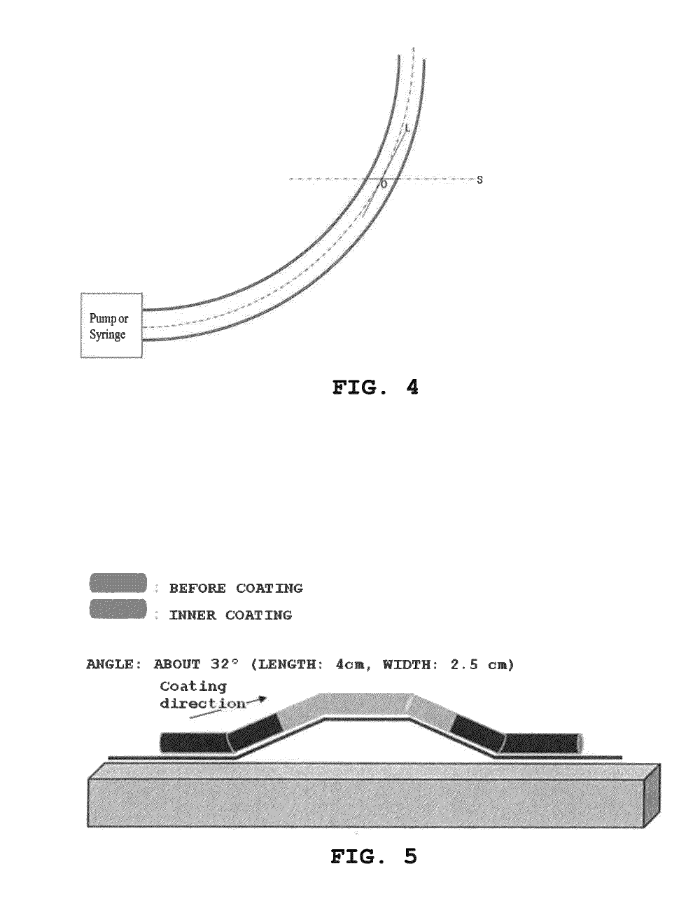

FIG. 4 shows a method of forming a coating layer including a bioactive material on a part of inner surface of both ends of a cylindrical structure. As shown in FIG. 4, a cylindrical structure is formed into a J-shaped cylindrical structure, a coating solution is perfused from the lower end of the J-shaped cylindrical structure to the upper portion thereof higher than the predetermined level (line S) using a solution supply unit, for example, a pump or a syringe, and then the coating solution perfused in the J-shaped cylindrical structure is discharged after predetermined time, thereby coating a part of the inner surface of one or more of both ends of the J-shaped cylindrical structure. In this case, it is preferred that the angle LOS be 30˜90°.

For example, each of the cylindrical structures shown in FIGS. 2B and 2C may be manufactured by perfusing a mixed solution containing a drug into the cylindrical structure and then coating a part of inner surface of one or more of both ends of the cylindrical structure with a mixed solution including a polymer for drug release control and a bioactive material using “J dipping” or “L dipping”.

Further, each of the cylindrical structures shown in FIGS. 2d and 2e may be manufactured by coating a part of inner surface of one or more of both ends of the cylindrical structure with a mixed solution including a polymer for drug release control and a bioactive material using “J dipping” or “L dipping” and then coating the outer surface of the cylindrical structure with a drug using spraying.

Further, each of the cylindrical structures shown in FIGS. 2f, 2g and 2h may be manufactured by perfusing a mixed solution containing a drug into the cylindrical structure, coating a part of inner surface of one or more of both ends of the cylindrical structure with a mixed solution including a polymer for drug release control and a bioactive material using “J dipping” or “L dipping” and then coating the outer surface of the cylindrical structure with a drug using spraying.

The cylindrical body having a lumen to be implanted into the human body according to the present invention may be configured such that it is partially or entirely connected to a blood vessel to allow blood to flow.

The cylindrical structure according to an embodiment of the present invention can be used as a tube for hemodialysis or an artificial blood vessel, which is connected with an artery or a vein.

The cylindrical structure according to another embodiment of the present invention can be used as an artificial blood vessel or an artificial lymph vessel by connecting this cylindrical structure with a blood vessel or lymph vessel, which is a circulation channel in the human body.

The cylindrical structure according to still another embodiment of the present invention can be used in diseases such as critical limb ischemia and the like, and can be used as an alternative blood vessel such as a coronary artery bypass graft (CABG) or the like as well as a blood vessel approach channel.

### Mode for Invention

Hereinafter, the present invention will be described in more detail with reference to the following Examples. These Examples are set forth to illustrate the present invention, and the scope of the present invention is not limited thereto.

### EXAMPLE 1

### Manufacture of Artificial Blood Vessel Coated with Paclitaxel

15 mg of PLGA and 125 mg of paclitaxel were put into a conical tube, dissolved in 4.5 mL of acetone, and then mixed with 0.5 mL of distilled water to prepare a coating solution in which 3 mg/mL of PLGA and 25 mg/mL of paclitaxel are dissolved in 90% acetone.

As shown in FIG. 5, an e-PTFE-made artificial blood vessel was inclined at an angle of about 32° (length: 4 cm, width: 2.5 cm) to a base plate, and then a peristaltic pump was connected to one end of the artificial blood vessel to slowly perfuse the coating solution into the artificial blood vessel for 1 minute. After 1 minute, the coating solution perfused into the artificial blood vessel in the opposite direction was discharged. Then, the coating solution was dried white injecting nitrogen gas into the artificial blood vessel for 5 minutes. The other end of the artificial blood vessel was also processed in the same manner. The artificial blood vessel coated with a drug was completely dried in vacuum for a day. As a result, an artificial blood vessel, both ends of which are provided therein with cylindrical structures having a length of 3 cm and coated with a drug in an amount of 0.44 μg/mm², was obtained.

### COMPARATIVE EXAMPLE 1

An e-PTFE-made artificial blood vessel was coated with paclitaxel over the entire inner and outer surface in an amount of 0.51 μg/mm² by dipping.

### COMPARATIVE EXAMPLE 2

A paclitaxel-dissolved 90% acetone solution flowed through an e-PTFE-made artificial blood vessel to coat the inner surface of a cylindrical structure with paclitaxel in an amount of 0.69 μg/mm².

### COMPARATIVE EXAMPLE 3

A paclitaxel-dissolved 90% acetone solution flowed through an e-PTFE-made artificial blood vessel to coat the inner surface of a cylindrical structure with paclitaxel in an amount of 0.22 μg/mm².

### TEST EXAMPLE

### Evaluation of Drug Release Patterns of Artificial Blood Vessels

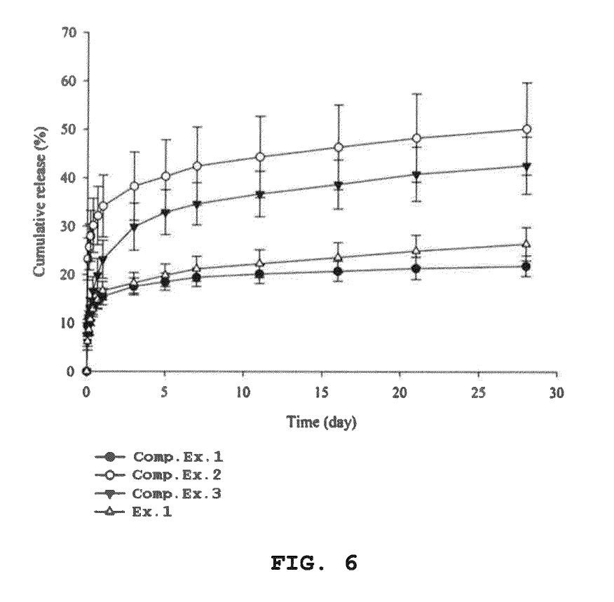

FIG. 6 is a graph comparing the drug release patterns of artificial blood vessels of Example 1 and Comparative Examples 1 to 3.

As shown in FIG. 6, it can be ascertained that the drug release rate of the artificial blood vessel of Example 1, which is coated with both paclitaxel and PLGA, is decreased compared to that of the artificial blood vessels of Comparative Examples 2 and 3, each of which is coated with only paclitaxel. Therefore, it can be ascertained that, when an artificial blood vessel coated with both drug and polymer is implanted into the human body, the drug release rate thereof can be reduced, thus enabling long-term drug release.

Consequently, when at least one part of inner surface of both ends of an artificial blood vessel is coated with a drug and a polymer, a drug can be slowly released for a long period of time while reducing toxicity, and thus neointimal hyperplasia occurring in the anastomosis site of a blood vessel can be effectively controlled.

1. A cylindrical structure having a lumen to be implanted into a human body, comprising:
a coating layer formed on at least one part of an inner surface of either or both ends of the cylindrical structure,
wherein the coating layer includes a polymer for drug release control and a bioactive material.

2. The cylindrical structure of claim 1, wherein the cylindrical structure is entirely or partially connected with a blood vessel to allow blood to flow therethrough.

3. The cylindrical structure of claim 1, wherein the cylindrical structure is made of e-PTFE (expanded polytetrafluoroethylene), polyethylene terephthalate, polyurethane, polyamide, polyester or polyolefin.

4. The cylindrical structure of claim 1, wherein the coating layer is formed by applying a mixed solution including a polymer for drug release control and a bioactive material onto inner surfaces of both ends of the cylindrical structure.

5. The cylindrical structure of claim 4, wherein the length of the coating layer from both ends of the cylindrical structure is 2˜15 cm.

6. The cylindrical structure of claim 1, wherein the bioactive material is one or a mixture of two or more selected from the group consisting of paclitaxel, methotrexate, rapamycin, tacrolimus, cyclosporine A. dexamethasone, dexamethasone phosphate, and dexamethasone acetate.

7. The cylindrical structure of claim 4, wherein the capacity of the bioactive material included in the cylindrical structure per unit area is 0.1˜3 μg/mm².

8. The cylindrical structure of claim 4, wherein the polymer for drug release control is collagen, cellulose acetate, hyaluronic acid, polylactic acid, polyglycolic acid, polyanhydride, polycaprolactone or a copolymer or compound thereof.

9. The cylindrical structure of claim 4, wherein the concentration of the polymer for drug release control included in the mixed solution is 0.01˜10 mg/mL (w/v).

10. The cylindrical structure of claim 4, wherein the mixed solution further includes a mixed solvent in which solvents having different penetrability to the cylindrical structure from each other are mixed.

11. The cylindrical structure of claim 10, wherein the mixed solvent is configured such that acetone and water are mixed at a ratio of 7:3˜9:1.

12. The cylindrical structure of claim 1, wherein a second coating layer including a bioactive material and a drug for inflammatory reaction control is additionally formed on an inner surface of the cylindrical structure, the inner surface not being provided with the coating layer.

13. The cylindrical structure of claim 1, wherein a second coating layer including a bioactive material and a drug for inflammatory reaction control is additionally formed on an outer surface of the cylindrical structure.

14. The cylindrical structure of claim 1, wherein a second coating layer including a bioactive material and a drug for inflammatory reaction control is additionally formed on an inner surface of the cylindrical structure, the inner surface not being provided with the coating layer, and a third coating layer including a bioactive material and a drug for inflammatory reaction control is additionally formed on an outer surface of the cylindrical structure.

15. The cylindrical structure of claim 1, wherein a coating layer including a polymer for drug release control and a bioactive material is formed on an entire inner surface of the cylindrical structure.

16. The cylindrical structure of claim 1, wherein a coating layer is at least partially formed on an inner surface of either or both ends of the cylindrical structure, and the coating layer includes a bioactive material.

17. A method of manufacturing a cylindrical structure having a lumen to be implanted into a human body of claim 1, comprising the step of forming a coating layer using one or more selected from the group consisting of perfusion, spraying and dipping.

18. The method of claim 17, comprising the steps of: forming the cylindrical structure into a J-shaped or L-shaped cylindrical structure;
upwardly perfusing a coating solution from one end of the J-shaped or L-shaped cylindrical structure to the other end thereof using a solution supply unit; and discharging the coating solution perfused in the J-shaped or L-shaped cylindrical structure after predetermined time to coat a part of the inner surface of one or more of both ends of the J-shaped or L-shaped cylindrical structure.

19. The method of claim 18, comprising the steps of: forming the cylindrical structure into a J-shaped cylindrical structure; perfusing a coating solution from the lower end of the J-shaped cylindrical structure to the upper portion thereof higher than the predetermined level using a solution supply unit; and discharging the coating solution perfused in the J-shaped cylindrical structure after predetermined time to coat a part of the inner surface of one or more of both ends of the J-shaped cylindrical structure, wherein the J-shape cylindrical structure is inclined at an angle of 30˜90° at the predetermined level,

20. The method of claim 17, comprising the steps of: perfusing a coating solution including a drug into the cylindrical structure; forming the cylindrical structure into a J-shaped or L-shaped cylindrical structure; upwardly perfusing a coating solution including a polymer for drug release control and a bioactive material from one end of the J-shaped or L-shaped cylindrical structure to the other end thereof; and discharging the coating solution perfused in the J-shaped or L-shaped cylindrical structure to the lower end of the J-shaped or L-shaped cylindrical structure after predetermined time.

21. The method of claim 17, comprising the steps of: forming the cylindrical structure into a J-shaped or L-shaped cylindrical structure;
upwardly perfusing a coating solution including a polymer for drug release control and a bioactive material from one end of the J-shaped or L-shaped cylindrical structure to the other end thereof; discharging the coating solution perfused in the J-shaped or L-shaped cylindrical structure to the lower end thereof after predetermined time to coat a part of the inner surface of one or more of both ends of the J-shaped or L-shaped cylindrical structure; and coating the outer surface of the J-shaped or L-shaped cylindrical structure with the coating solution using spraying or dipping.

22. The method of claim 17, comprising the steps of: perfusing a coating solution including a drug into the cylindrical structure; forming the cylindrical structure into a J-shaped or L-shaped cylindrical structure; upwardly perfusing a coating solution including a polymer for drug release control and a bioactive material from one end of the J-shaped or L-shaped cylindrical structure to the other end thereof; discharging the coating solution perfused in the J-shaped or L-shaped cylindrical structure to the lower end thereof after predetermined time to coat a part of the inner surface of one or more of both ends of the J-shaped or L-shaped cylindrical structure; and coating the outer surface of the J-shaped or L-shaped cylindrical structure with the coating solution using spraying or dipping.

23. The method of claim 17, wherein a mixed solution including a polymer for drug release control, a penetrable solvent and a drug is perfused into the cylindrical structure to coat the entire inner surface of the cylindrical structure with this mixed solution.

24. The method of claim 17, wherein the cylindrical structure is a tube for hemodialysis or an artificial blood vessel.
